(19) United States
(12) Patent Application Publication
(10) Pub. No.: US 2025/0320463 A1
(43) Pub. Date: Oct. 16, 2025

(54) THREE-DIMENSIONAL STRUCTURE, METHOD OF MODELING BRONCHIOLITIS OBLITERANS SYNDROME, AND ASSOCIATED METHODS

(71) Applicants: Georgia Tech Research Corporation, Atlanta, GA (US); Emory University, Atlanta, GA (US)

(72) Inventors: Shuichi Takayama, Atlanta, GA (US); Ji-Hoon Lee, Atlanta, GA (US); Kirsten Williams, Atlanta, GA (US)

(21) Appl. No.: 19/175,737

(22) Filed: Apr. 10, 2025

Related U.S. Application Data
(60) Provisional application No. 63/632,213, filed on Apr. 10, 2024.

(51) Int. Cl. C12N 5/071; C12N 5/077

(52) U.S. Cl. CPC C12N 5/0688; C12N 5/0656; C12N 2502/11; C12N 2503/04; C12N 2513/00

(57) ABSTRACT

The present disclosure relates to a three-dimensional structure including an inverted co-culture having an interior layer comprising a plurality of cells of a first cell type; an opposing exterior layer comprising a plurality cells of a second cell type; and a basement membrane matrix positioned between the interior layer and the exterior layer, wherein the three-dimensional structure has a diameter, and wherein said diameter is capable of being altered by an alloimmune reaction that disrupts the plurality of cells of the second cell type and leads to contraction of the plurality of cells of the second cell type. Also disclosed is a method of modeling Bronchiolitis Obliterans Syndrome, a method for assessing presence of or risk of developing a physiological condition, a method of identifying one or more biomarkers of a disease, and a method of a method of making a three-dimensional structure.

### CROSS-REFERENCE TO RELATED APPLICATIONS

This application claims the benefit of U.S. Provisional Application Ser. No. 63/632,213, filed on Apr. 10, 2024, which is incorporated herein by reference in its entirety as if fully set forth below.

### GOVERNMENT LICENSE RIGHTS

This invention was made with government support under HL136141 awarded by the National Institutes of Health. The government has certain rights in the invention.

### FIELD OF THE DISCLOSURE

The various embodiments of the present disclosure relate generally to a three-dimensional structure, a method of modeling Bronchiolitis Obliterans Syndrome (BOS), and associated methods.

### BACKGROUND

Chronic pulmonary graft-versus-host disease (GVHD) is a morbid and deadly complication of allogeneic hematopoietic cell transplantation (HCT). Schwarer et al., “A Chronic Pulmonary Syndrome Associated With Graft-versus-host Disease After Allogeneic Marrow Transplantation,” Transplantation 54(6):1002-1008 (1992) and Yanik and Cooke, “The Lung as a Target Organ of Graft-Versus-Host Disease,” Seminars in Hematology 43:42-52 (2006). A specific manifestation of this condition, known as Bronchiolitis Obliterans Syndrome (BOS), affects a notable percentage—between 5-12%—of survivors after allogenic HCT. Williams et al., “Bronchiolitis Obliterans After Allogeneic Hematopoietic Stem Cell Transplantation,” JAMA 302:306-314 (2009); Chien et al., “Bronchiolitis Obliterans Syndrome After Allogeneic Hematopoietic Stem Cell Transplantation—An Increasingly Recognized Manifestation of Chronic Graft-versus-Host Disease,” Biology of Blood and Marrow Transplantation 16(1 Suppl):5106-5114 (2010); Au et al., “Bronchiolitis Obliterans Syndrome Epidemiology after Allogeneic Hematopoietic Cell Transplantation,” Biology of Blood and Marrow Transplantation 17:1072-1078 (2011); Hildebrandt et al., “Diagnosis and Treatment of Pulmonary Chronic GVHD: Report From the Consensus Conference on Clinical Practice in Chronic GVHD,” Bone Marrow Transplant 46: 1283-1295 (2011); Williams, K. M., “How I Treat Bronchiolitis Obliterans Syndrome After Hematopoietic Stem Cell Transplantation,” Blood 129:448-455 (2017); Holland et al., “Bronchiolitis Obliterans in Bone Marrow Transplantation and Its Relationship to Chronic Graft-v-Host Disease and Low Serum IgG,” Blood 72:621-627 (1988); and Archer et al., “Interstitial Lung Diseases After Hematopoietic Stem Cell Transplantation: New Pattern of Lung Chronic Graft-versus-host Disease?,” Bone Marrow Transplant 58:87-93 (2023), all of which are hereby incorporated by reference in their entirety. BOS, characterized by airway inflammation leading to progressive airway fibrosis (Gabbay et al., “Post-lung Transplant Bronchiolitis Obliterans Syndrome (BOS) is Characterized by Increased Exhaled Nitric Oxide Levels and Epithelial Inducible Nitric Oxide Synthase,” Am. J. Respir. Crit. Care. Med. 162:2182-2187 (2000)), presents with a spectrum of symptoms, including, but not limited to, air trapping, progressive dyspnea, recurrent infections, and a marked decline in quality of life, culminating in fatality in approximately 50% of patients at 5 years (Bergeron and Cheng, “Bronchiolitis Obliterans Syndrome and Other Late Pulmonary Complications After Allogeneic Hematopoietic Stem Cell Transplantation,” Clinics in Chest Medicine 38607-621 (2017)). The complexity of BOS pathogenesis, compounded by its insidious presentation, poor prognosis, and the challenge of accessing affected tissues, has hindered comprehensive research efforts. Williams, K. M., “How I Treat Bronchiolitis Obliterans Syndrome After Hematopoietic Stem Cell Transplantation,” Blood 129:448-455 (2017). Despite extensive exploration into clinical management strategies (Ling et al., “Azithromycin Partially Mitigates Dysregulated Repair of Lung Allograft Small Airway Epithelium,” Transplantation 104(6):1166-1176 (2020) and Williams et al., “Fluticasone, Azithromycin, and Montelukast Treatment for New-Onset Bronchiolitis Obliterans Syndrome after Hematopoietic Cell Transplantation,” Biology of Blood and Marrow Transplantation 22:710-716 (2016)), the rarity of the disease has impeded the establishment of standardized diagnostic protocols (Jagasia et al., “National Institutes of Health Consensus Development Project on Criteria for Clinical Trials in Chronic Graft-versus-Host Disease: I. The 2014 Diagnosis and Staging Working Group Report,” Biology of Blood and Marrow Transplantation 21:389-401 (2015)). Furthermore, most patients remain asymptomatic until advanced stages, by which time irreversible lung function decline has often occurred. Williams, K. M., “How I Treat Bronchiolitis Obliterans Syndrome After Hematopoietic Stem Cell Transplantation,” Blood 129:448-455 (2017). While animal models of BOS have been developed (Panoskaltsis-Mortari et al., “A New Murine Model for Bronchiolitis Obliterans Post-Bone Marrow Transplant,” Am. J. Respir. Crit. Care. Med. 176:713-723 (2007) and Swatek et al., “Depletion of Airway Submucosal Glands and TP63+KRT5+ Basal Cells in Obliterative Bronchiolitis,” Am. J. Respir. Crit. Care. Med. 197:1045-1057 (2018)), disparities in lung physiology between humans and non-human models result in an incomplete representation of the disease. In addition, while BOS after HCT is rare, a similar disease, BOS after lung transplant is more common, with equally poor survival without re-transplantation. Hence, there is an urgent need for an enhanced model system to study BOS. Such improved models hold potential for unravelling the mechanisms underlying BOS and facilitating the development of targeted interventions to improve patient outcomes.

Following a recent initiative by the U.S. Food and Drug Administration (FDA) aimed at eliminating mandatory animal testing in drug research and development process (S.5002—117th Congress (2021-2022): FDA Modernization Act 2.0. (September 2022)), there has been a notable increase in interest surrounding 3D in vitro systems. This surge in interest is driven by the recognition of the potential for these systems to offer enhanced physiological relevance. Organoids, specifically, have taken center stage due to their remarkable ability to mimic the complexity of human tissues and organs. Rossi et al., “Progress and Potential in Organoid Research,” Nat. Rev. Genet. 19:671-687 (2018). An organoid culture method has been described based on minimal ECM scaffolding, resulting in ECM-encapsulating organoids with stable apical-out morphology (Parigoris et al., “Cancer Cell Invasion of Mammary Organoids With Basal-in Phenotype,” Advanced Healthcare Materials 10(4):e2000810 (2021); Parigoris et al., “Extended Longevity Geometrically Inverted Proximal Tubule Organoids,” Biomaterials 290:121828 (2022); Lee et al., “Development of Robust Antiviral Assays Using Relevant Apical-Out Human Airway Organoids,” bioRxiv 12:2024 (2024); Lee et al., “High Throughput Formation and Image-based Analysis of Basal-in Mammary Organoids in 384-well Plates,” Scientific Reports 12(1):317 (2022); Mertz et al., “Triple-negative Breast Cancer Cells Invade Adipocyte/preadipocyte-encapsulating Geometrically Inverted Mammary Organoids,” Integrative Biology 15:zyad004 (2023)). Most recently, the technique was exploited to culture apical-out human primary airway organoids for SARS-CoV-2 infection and antiviral screening. Lee et al., “Development of Robust Antiviral Assays Using Relevant Apical-Out Human Airway Organoids,” bioRxiv 12:2024 (2024). In addition to its unique demonstration, this approach offers several proven benefits, including long-term stability, ease of interpretation, and potential for standardization.

There is a need to develop robust systems and methods for assessing cancer, bacterial, or viral invasion as well as drug screening platforms using organoids that allow easier access to the apical surface.

Bronchiolitis Obliterans Syndrome (BOS) remains a challenging condition in the context of chronic graft-versus-host disease (cGVHD) post-hematopoietic cell transplantation (HCT). Despite efforts to understand its pathogenesis, several hurdles impede progress, including the inadequacy of existing animal models, delayed diagnosis, and limited access to affected anatomical sites. Murine models, while available, often fail to fully replicate the disease phenotype due to cellular differences between mice and humans, resulting in poor penetrance and incomplete representation of clinical features.

Accordingly, there is a need for organoids and three-dimensional structures that allow for the study of this and similar pathophysiologies.

### BRIEF SUMMARY

A first aspect of the present disclosure provides a three-dimensional structure. The three-dimensional structure includes an inverted co-culture including: an interior layer comprising a plurality of cells of a first cell type; an opposing exterior layer comprising a plurality cells of a second cell type; and a basement membrane matrix positioned between the interior layer and the exterior layer, wherein the three-dimensional structure has a diameter, and wherein said diameter is capable of being altered by an alloimmune reaction that disrupts the plurality of cells of the second cell type and leads to contraction of the plurality of cells of the second cell type.

In any of the embodiments disclosed herein, the first cell type of the three-dimensional structure comprises stromal cells.

In any of the embodiments disclosed herein, the stromal cells in the three-dimensional structure comprise fibroblasts.

In any of the embodiments disclosed herein, the second cell type in the three-dimensional structure comprises epithelial cells.

In any of the embodiments disclosed herein, the epithelial cells are airway epithelial cells.

In any of the embodiments disclosed herein, the epithelial cells are selected from ciliated cells, secretory cells, ionocytes, basal cells, submucosal gland cells, club cells, Type I and Type II alveolar cells, hillock cells, or any combination thereof.

In any of the embodiments disclosed herein, the epithelial cells comprise lung cells, bronchial cells, tracheal cells, alveolar cells, mammary cells, kidney cells, bladder cells, corneal cells, prostate cells, renal cells, vaginal cells, cervical cells, intestinal cells, or combinations thereof.

In any of the embodiments disclosed herein, the three-dimensional structure comprises an interior chamber.

In any of the embodiments disclosed herein, the interior chamber comprises a plurality of cells of a third cell type.

In any of the embodiments disclosed herein, the third cell type of cells comprises stromal cells, mesenchymal cells, chondrocytes, osteoblasts, adipocytes, myocytes, pericytes, endothelial cells, or any combination thereof.

In any of the embodiments disclosed herein, the stromal cells comprise fibroblasts, myofibroblasts, adipocytes, fibrocytes, pericytes, mesenchymal stem cells, macrophages, mast cells, lymphocytes, neutrophils, other leukocytes, endothelial cells, smooth muscle cells, or any combination thereof.

In any of the embodiments disclosed herein, wherein the diameter of the three-dimensional structure is between about 100 micrometers and about 5 mm.

In any of the embodiments disclosed herein, when the diameter is altered, the diameter is reduced by between about 10% and about 60% resulting in a second diameter, or the diameter is reduced by a decrease in roundness resulting in a second diameter, or a combination thereof.

In any of the embodiments disclosed herein, the ratio amount of the second cell type and the first cell type is between 100:1 and 1:1.

In any of the embodiments disclosed herein, the first cell type, the second cell type, and/or the third cell type comprise human cells.

Another aspect of the present disclosure provides a method of modeling Bronchiolitis Obliterans Syndrome (BOS). The method includes providing the three-dimensional structure described herein, and exposing the exterior layer of the three-dimensional structure to a plurality of blood cells, under conditions effective to model Bronchiolitis Obliterans Syndrome.

In any of the embodiments disclosed herein, the method is carried out after a lung transplant or after a bone marrow transplant.

In any of the embodiments disclosed herein, the first cell type comprises stromal cells.

In any of the embodiments disclosed herein, the stromal cells comprise fibroblasts.

In any of the embodiments disclosed herein, the second cell type comprises epithelial cells.

In any of the embodiments disclosed herein, the epithelial cells are airway epithelial cells.

In any of the embodiments disclosed herein, the epithelial cells are selected from ciliated cells, secretory cells, ionocytes, basal cells, submucosal gland cells, club cells, Type I and Type II alveolar cells, hillock cells, or any combination thereof.

In any of the embodiments disclosed herein, the epithelial cells comprise lung cells, bronchial cells, tracheal cells, alveolar cells, mammary cells, kidney cells, bladder cells, corneal cells, prostate cells, renal cells, vaginal cells, cervical cells, intestinal cells, or combinations thereof.

In any of the embodiments disclosed herein, the three-dimensional structure comprises an interior chamber.

In any of the embodiments disclosed herein, the interior chamber comprises a plurality of cells of a third cell type.

In any of the embodiments disclosed herein, the third cell type of cells comprises stromal cells, mesenchymal cells, chondrocytes, osteoblasts, adipocytes, myocytes, pericytes, endothelial cells, or any combination thereof.

In any of the embodiments disclosed herein, the stromal cells comprise fibroblasts, myofibroblasts, adipocytes, fibrocytes, pericytes, mesenchymal stem cells, macrophages, mast cells, lymphocytes, neutrophils, other leukocytes, endothelial cells, smooth muscle cells, or any combination thereof.

In any of the embodiments disclosed herein, the first cell type, the second cell type, and/or the third cell type comprise human cells.

In any of the embodiments disclosed herein, the plurality of blood cells comprises one or more of hematopoietic stem cells, hematopoietic circulating cells, or other leukocytes, peripheral blood stem cells (PBSCs), patient-derived cells, engineered cells, peripheral blood mononuclear cells (PBMCs), isolated lymphocytes, chimeric antigen receptor (CAR)-T cells, neutrophils, or monocytes.

In any of the embodiments disclosed herein, the method is carried out in vitro.

In any of the embodiments disclosed herein, the method further includes providing one or more healthy lung cells and one or more healthy donor HLA-mismatched immune cells.

In any of the embodiments disclosed herein, the method further includes identifying one or more of a plurality of disease progression phases.

In any of the embodiments disclosed herein, the method further includes testing a treatment during at least one of the one or more of the plurality of disease progression phases.

In any of the embodiments disclosed herein, the treatment is selected from one or more of a cytokine blockade, one or more of a specific cell population blockade, one or more of an agent to aid in epithelia repair, or any combination thereof.

Another aspect of the present disclosure provides a method for assessing presence of or risk of developing a physiological condition. The method includes providing the three-dimensional structure described herein, and exposing the exterior layer of the three-dimensional structure to a plurality of blood cells, under conditions effective to assess presence of or risk of developing a physiological condition.

In any of the embodiments disclosed herein, the plurality of blood cells comprises one or more of hematopoietic stem cells, hematopoietic circulating cells, or other leukocytes, peripheral blood stem cells (PBSCs), patient-derived cells, engineered cells, peripheral blood mononuclear cells (PBMCs), isolated lymphocytes, chimeric antigen receptor (CAR)-T cells, neutrophils, or monocytes.

In any of the embodiments disclosed herein, the plurality of blood cells are present in the amount of between about 300 to about 300,000 cells.

In any of the embodiments disclosed herein, the physiological condition comprises a recapitulation of one or more diseases involving an immune response.

In any of the embodiments disclosed herein, the physiological condition comprises one or more diseases involving epithelial-stromal-blood cells.

In any of the embodiments disclosed herein, the method further includes varying degrees of human leukocyte matching of the three-dimensional structure for at least one of the cells of the first cell type, the second cell type, and/or the blood cells.

In any of the embodiments disclosed herein, the varying degrees of human leukocyte matching for at least one of the cells of the three-dimensional structure comprises varying the degree of antigen matching.

In any of the embodiments disclosed herein, the varying degrees of human leukocyte matching for at least one of the cells of the three-dimensional structure comprises varying the degree of protein level.

In any of the embodiments disclosed herein, the varying degrees of human leukocyte matching comprises fully matching human leukocyte antigens (HLA) between one or more cells of the second cell type and one or more blood cells.

In any of the embodiments disclosed herein, the varying degrees of matching comprises tuning human leukocyte antigens (HLA) mismatch between one or more cells of the second cell type and one or more blood cells.

In any of the embodiments disclosed herein, the method further includes culturing a plurality of the three-dimensional structure in a plurality of wells in a single-organoid-per-well format; and manipulating a growth factor level in the plurality of wells.

In any of the embodiments disclosed herein, the method further comprises isolating RNA from the single three-dimensional structure.

In any of the embodiments disclosed herein, the method further includes sequencing RNA isolated from the single three-dimensional structure.

In any of the embodiments disclosed herein, the method further includes harvesting a supernatant from the plurality of wells; and assaying the supernatant.

Another aspect of the present disclosure provides a method of identifying one or more biomarkers of a disease. The method includes providing the three-dimensional structure described herein; exposing the exterior layer of the three-dimensional structure to a plurality of blood cells; and identifying one or more biomarkers of a disease.

In any of the embodiments disclosed herein, the method further includes diagnosing or prognosing a disease based on the presence or absence of one or more biomarkers of disease.

In any of the embodiments disclosed herein, the biomarkers of disease are selected from the group consisting of interleukin-6 (IL-6), c-reactive protein (CRP), tumor necrosis factor-alpha (TNF-α), donor-derived cell-free DNA (dd-cfDNA), lymphocyte count, neutrophil count, surfactant proteins (SP-A, SP-D), krebs von den Lungen-6 (KL-6), matrix metalloproteinases (MMP), transforming growth factor-beta (TGF-β), procalcitonin (PCT), galactomannan, cytomegalovirus (CMV) DNA, pathogen-specific PCR, b-type natriuretic peptide (BNP), lactate dehydrogenase (LDH), total protein, albumin, erythrocyte sedimentation rate (ESR), and fibrinogen.

In any of the embodiments disclosed herein, the method further includes identifying targetable pathways for treatment of the disease.

In any of the embodiments disclosed herein, the targetable pathway for treatment of the disease is mechanistic target of rapamycin (mTOR) pathway in T cells.

In any of the embodiments disclosed herein, the method further includes testing a response of the one or more biomarkers of a disease in response to one or more interventions.

In any of the embodiments disclosed herein, the one or more interventions is a TNF-alpha blockade.

Another aspect of the present disclosure provides a method of making a three-dimensional structure. The method includes providing a plurality of cells of a first cell type; providing a plurality cells of a second cell type; providing a basement membrane matrix material; and co-culturing the plurality of cells of the first type, the plurality of cells of the second cell type, and the basement membrane matrix material under conditions effective to form the three-dimensional structure described herein.

BOS remains a rare but challenging condition in the context of cGVHD post-HCT. Despite efforts to understand its pathogenesis, several hurdles impede progress, including the inadequacy of existing animal models, delayed diagnosis, and limited access to affected anatomical sites. Murine models, while available, often fail to fully replicate the disease phenotype due to cellular differences between mice and humans, resulting in poor penetrance and incomplete representation of clinical features. Disclosed herein is a novel co-culture model utilizing human primary cells, aimed at better recapitulating the clinical complexity of BOS. This model incorporates key human cell populations implicated in the disease, including airway epithelial cells, fibroblasts, and hematopoietic cells derived from PBSCs, which have been linked to cGVHD and BOS. By adjusting HLA matching between epithelial and immune cells, a closer resemblance to the clinical condition is achieved, evidenced by epithelial injury, fibroblast expansion, and extrusion observed through immunofluorescence analysis. RNA sequencing further elucidated gene expression changes, highlighting pathways relevant to BOS pathogenesis. Overall, the tri-culture organoid model disclosed herein represents a significant advancement in BOS research, offering a physiologically relevant platform for mechanistic studies and therapeutic discovery, with potential for broad translational impact.

In this disclosure, the physiological relevance of the airway organoid model is enhanced by incorporating stromal and immune cells. First, a co-culture system comprising human primary airway epithelial cells and fibroblasts was optimized, where possible with matched human leukocyte antigens (HLA), to recapitulate pre-HCT recipient airways. Next, mobilized peripheral blood stem cells (PBSCs) isolated from two healthy adult donors, with varying degrees of HLA mismatch were added to the co-culture organoids, mimicking post-allogenic HCT responses. The outward facing epithelial layer enabled direction interactions between the epithelial cells and PBSCs, akin to the interactions in vivo. After exposure, epithelial disruption occurred with ensuing fibroblast extrusion to cover the defect, confirmed by microscopic analysis, indicative of BOS intraluminal fibrosis. In summary, the tri-culture approach described herein recapitulates key physiological architecture of the airway, mimicking epithelial-fibroblast structures alongside PBSC-mediated immune responses, thereby offering a comprehensive in vitro platform for studying BOS pathophysiology and potential therapeutic interventions.

These and other aspects of the present disclosure are described in the detailed description below and the accompanying drawings. Other aspects and features of embodiments will become apparent to those of ordinary skill in the art upon reviewing the following description of specific, exemplary embodiments in concert with the drawings. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIGS. 1A-1E show optimization of NHBE-IMR90 organoids. FIG. 1A shows that seeding IMR90 fibroblasts only as organoids results in a smaller diameter, with their cores filled with fibroblasts (red, vimentin). FIG. 1B shows that NHBE-only organoids exhibit hollow cores with minimal vimentin IF signals. FIGS. 1C and 1D show that the co-seeding of NHBE and IMR90 at 6:1 and 2:1 ratio, respectively, results in the formation of organoids with fibroblasts within the core. FIG. 1E depicts bar graphs showing vimentin IF levels of with differing epithelial-fibroblast seeding ratios. Lines above the bar graphs indicate significance levels from Tukey's post hoc tests with Bonferroni multiple comparison corrections. ** represents p<0.01, and **** represents p<0.0001. Scale bars, 200 μm in FIGS. 1A-1D.

FIGS. 2A-2F show NHBE-NHLF organoid culture. FIG. 2A shows a schematic illustration of NHBE-NHLF organoids, composed of the airway cells on the outer layer, followed by basement membranes, and networks of fibroblasts in the core. FIGS. 2B and 2C show micrographs of the organoids at day 1 of culture with fluorescently labelled fibroblasts (red) for 6:1 and 2:1 ratio, respectively. FIG. 2D shows time-course culture of the organoids imaged using qOBM technique. FIGS. 2E and 2F show confocal imaging slice confirming the co-culture cells with the expected inverted polarity (red, vimentin; green, E-cad; blue, DAPI) for 6:1 and 2:1 ratio, respectively. Scale bars, 200 μm in FIGS. 2B-2F.

FIGS. 3A-3F show NHBE-IMR90 BOS organoid. FIGS. 3A, 3B, and 3C show time-course micrographs post-addition of PBSCs at three ten-fold doses (300, 3000, and 30000 cells, respectively). Epithelial damage is followed by fibroblast proliferation and differentiation. FIG. 3D shows IF staining of 3 representative BOS organoids (red, vimentin; green, E-cad; blue, DAPI). FIG. 3E shows % reduction in organoid size by day 5 of introducing PBSCs. Scale bars, 200 μm in FIGS. 3A-3C, and 100 μm in FIGS. 3D-3E. FIG. 3F shows a schematic of the co-culture of inner fibroblasts and outer epithelia that comprise the BOS organoid surrounded by the peripheral blood stem cells (left) and the subsequent immune mediated epithelial injury with fibroblast proliferation and extrusion into the epithelial defects.

FIGS. 4A-4F show additional time-course replicate data of NHBE+IMR90 organoids post PBSC introduction with a complete HLA mismatch. Tracking of organoids over a 5-day period following the addition of PBSCs at three doses: 300 (FIGS. 4A and 4B), 3000 (FIGS. 4C and 4D), and 30000 (FIGS. 4E and 4F) cells. Scale bars, 200 μm.

### DETAILED DESCRIPTION

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

As used in the specification and the appended claims, the singular forms “a,” “an” and “the” include plural referents unless the context clearly dictates otherwise.

Also, in describing the preferred exemplary embodiments, terminology will be resorted to for the sake of clarity. It is intended that each term contemplates its broadest meaning as understood by those skilled in the art and includes all technical equivalents that operate in a similar manner to accomplish a similar purpose.

Ranges can be expressed herein as from “about” or “approximately” one particular value and/or to “about” or “approximately” another particular value. When such a range is expressed, another exemplary embodiment includes from the one particular value and/or to the other particular value.

By “comprising” or “containing” or “including” is meant that at least the named compound, member, particle, or method step is present in the composition or article or method, but does not exclude the presence of other compounds, materials, particles, method steps, even if the other such compounds, material, particles, method steps have the same function as what is named.

Mention of one or more method steps does not preclude the presence of additional method steps or intervening method steps between those steps expressly identified. Similarly, it is also to be understood that the mention of one or more components in a device or system does not preclude the presence of additional components or intervening components between those components expressly identified.

The materials described as making up the various members of the invention are intended to be illustrative and not restrictive. Many suitable materials that would perform the same or a similar function as the materials described herein are intended to be embraced within the scope of the invention. Such other materials not described herein can include, but are not limited to, for example, materials that are developed after the time of the development of the invention.

A first aspect of the present disclosure provides a three-dimensional structure. The three-dimensional structure includes an inverted co-culture including: an interior layer comprising a plurality of cells of a first cell type; an opposing exterior layer comprising a plurality cells of a second cell type; and a basement membrane matrix positioned between the interior layer and the exterior layer, wherein the three-dimensional structure has a diameter, and wherein said diameter is capable of being altered by an alloimmune reaction that disrupts the plurality of cells of the second cell type and leads to contraction of the plurality of cells of the second cell type.

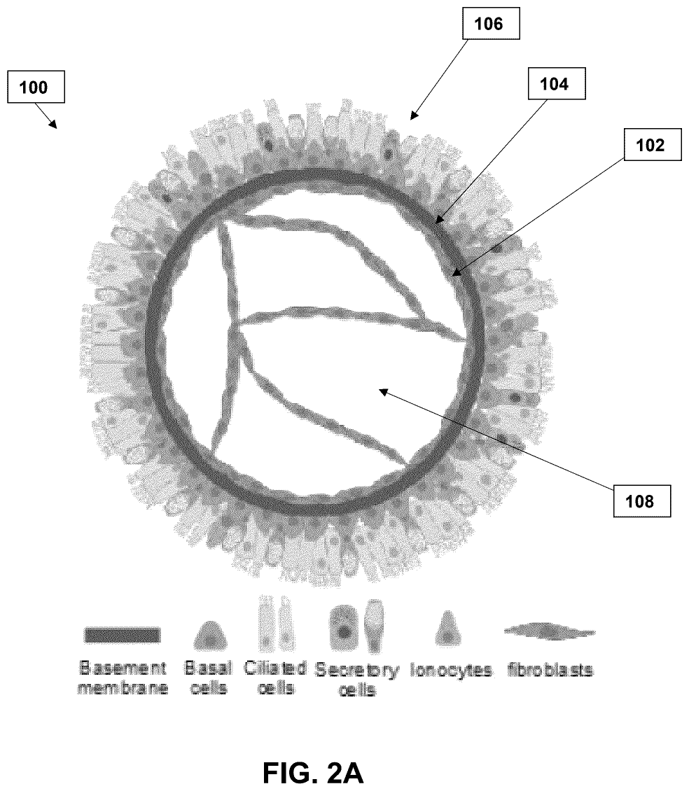

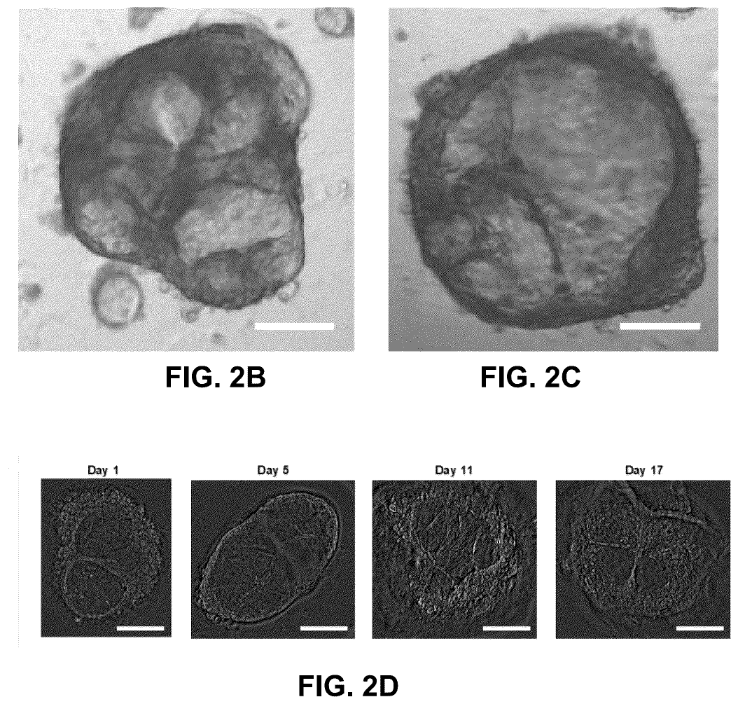

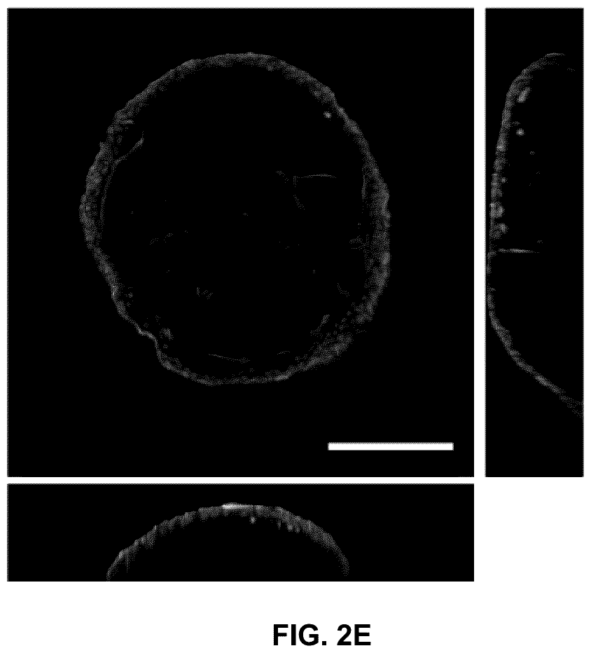

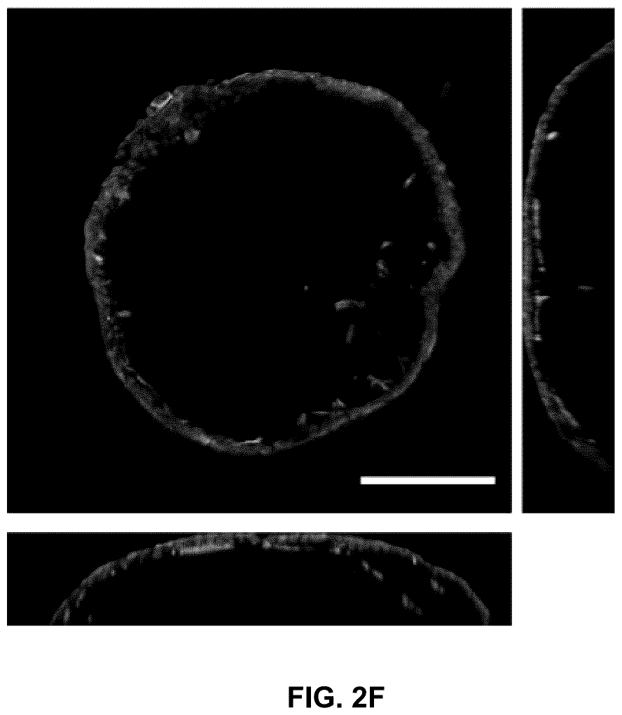

FIG. 2A shows an exemplary three-dimensional structure in accordance with the aspects and embodiments described herein. FIG. 2A provides an example apical-out, or basal-in, three-dimensional structure 100. In some embodiments, apical-out three-dimensional structure 100 can be inverted such that the interior layer 102 includes a plurality of cells of a first type, for example, stromal cells, or in some embodiments, more particularly fibroblasts, and is positioned to face inwardly on an interior surface and defining an interior chamber 108 of the three-dimensional structure 100.

The three-dimensional structure 100 further may further include, on an exterior surface and positioned outwardly, an exterior layer 106 that comprises a plurality of cells of a second cell type, for example, epithelial cells. The three-dimensional structure 100 further includes a basement membrane matrix 104 positioned between interior layer 102 and exterior layer 106. Interior layer 102, exterior layer 106, and/or basement membrane 104 may be a sheet-like type of matrix that provides the three-dimensional structure with cell and tissue support. In some embodiments, any or all of interior layer 102, exterior layer 106, and/or basement membrane 104 can be uniformly developed and fully encapsulate interior chamber 108. In some embodiments, any or all of interior layer 102, exterior layer 106, and/or basement membrane 104 can form one or more partial layers and have gaps along the layer around the interior chamber 108. As would be appreciated by those of skill in the art, the three-dimensional structure can be formed with or without a complete any or all of interior layer 102, exterior layer 106, and/or basement membrane 104 while still maintaining a spherical apical-out three-dimensional structure.

In some embodiments, basement membrane matrix 104 can be measured by integrin alpha-6 staining which can adhere to the basement membrane matrix 104. Additionally, or alternatively thereto, laminin-5 can be used as an indicator of the basement membrane matrix 104.

In the three-dimensional structure described herein, a first surface of interior layer 102 may interact with the interior chamber 108, basement membrane matrix 104, and/or opposing exterior layer 106 of the three-dimensional structure 100. In some embodiments, the interior layer 102 comprises a plurality of cells of a first cell type, and the first cell type of are stromal cells. In any of the embodiments disclosed herein, the stromal cells may be fibroblasts.

In the three-dimensional structure described herein, a first surface of opposing exterior layer 106 may interact with the interior layer 102, interior chamber 108, and/or basement membrane matrix 104 of the three-dimensional structure 100. In any of the embodiments disclosed herein, the second cell type in the three-dimensional structure comprises epithelial cells. In any of the embodiments disclosed herein, the epithelial cells may be airway epithelial cells. In any of the embodiments disclosed herein, the epithelial cells may include, but are not limited to, ciliated cells, secretory cells, ionocytes, basal cells, submucosal gland cells, club cells, Type I and Type II alveolar cells, hillock cells, or any combination thereof.

The plurality of cells of the second cell type can be on the external surface and interact with the environment of the three-dimensional structure 100. The second cell type can be made up of epithelium tissue. The second cell type can be arranged in a single layer of cells, (e.g., squamous, columnar, cuboidal, or specialized) and can also be stratified or pseudostratified and arranged in layers of two or more cells deep. Three-dimensional structure 100 can be either a monolayer or a multilayer. The epithelial cells can include but are not limited to lung cells, bronchial cells, tracheal cells, alveolar cells, mammary cells, kidney cells, bladder cells, corneal cells, prostate cells, renal cells, vaginal cells, cervical cells, intestinal cells, or combinations thereof. In some embodiments, the cells of the second cell type can further include connective tissue, muscle tissue, and/or nervous tissue.

In any of the embodiments disclosed herein, the ratio amount of the second cell type and the first cell type is between about 100:1 and about 1:1. For example, the ratio of the amount of the second cell type and the first cell type may be about 100:1, about 99:1, about 98:1, about 97:1, about 96:1, about 95:1, about 94:1, about 93:1, about 92:1, about 91:1 about 90:1, about 85:1, about 80:1, about 75:1, about 70:1, about 65:1, about 60:1, about 55:1, about 50:1, about 45:1, about 40:1, about 35:1, about 30:1, about 25:1, about 20:1, about 15:1, about 10:1, about 9:1, about 8:1, about 7:1, about 6:1, about 5:1, about 4:1, about 3:1, about 2:1, about 1:1, or any amount therebetween.

In any of the embodiments disclosed herein, the three-dimensional structure comprises an interior chamber. In any of the embodiments disclosed herein, the interior chamber may include a plurality of cells of a third cell type. The third cell type may be in the form of a connected network of cells of the third cell type. The third cell type of cells may include, but are not limited to stromal cells, mesenchymal cells, chondrocytes, osteoblasts, adipocytes, myocytes, pericytes, endothelial cells, or any combination thereof. The stromal cells may include fibroblasts, myofibroblasts, adipocytes, fibrocytes, pericytes, mesenchymal stem cells, macrophages, mast cells, lymphocytes, neutrophils, other leukocytes, endothelial cells, smooth muscle cells, or any combination thereof. In any of the embodiments disclosed herein, the first cell type, the second cell type, and/or the third cell type may be human cells.

The three-dimensional structure described herein are generally highly spherical (e.g., having circularity above about 0.80, above about 0.81, above about 0.82, above about 0.83, above about 0.84, above about 0.85, above about 0.86, above about 0.87, above about 0.88, above about 0.89, and/or above about 0.9). The spherical nature of the three-dimensional structure may be due to epithelial transport of ions, molecules, and fluids from the apical side to the basolateral side that stretches the epithelium. In any of the embodiments disclosed herein, the diameter of the three-dimensional structure may be between about 100 micrometers and about 5 millimeters. For example, the diameter of the three-dimensional structure may be between about 100 micrometers and about 4 millimeters, between about 100 micrometers and about 3 millimeters, between about 100 micrometers and about 2 millimeters, between about 100 micrometers and about 1 millimeters, between 1 millimeter and about 5 millimeters, between about 1 millimeter and about 4 millimeters, between about 1 millimeter and about 3 millimeters, between about 1 millimeter and about 2 millimeters, or any range therebetween. In various examples, the diameter of the of the three-dimensional structure may be about 100 micrometers, about 150 micrometers, about 200 micrometers, about 250 micrometers, about 300 micrometers, about 350 micrometers, about 400 micrometers, about 450 micrometers, about 500 micrometers, about 550 micrometers, about 600 micrometers, about 650 micrometers, about 700 micrometers, about 750 micrometers, about 800 micrometers, about 850 micrometers, about 900 micrometers, about 1,000 micrometers (or 1 millimeter), about 1 millimeter, about 1.5 millimeters, about 2.0 millimeters, about 2.5 millimeters, about 3.0 millimeters, about 3.5 millimeters, about 4.0 millimeters, about 4.5 millimeters, about 5.0 millimeters, or any amount therebetween.

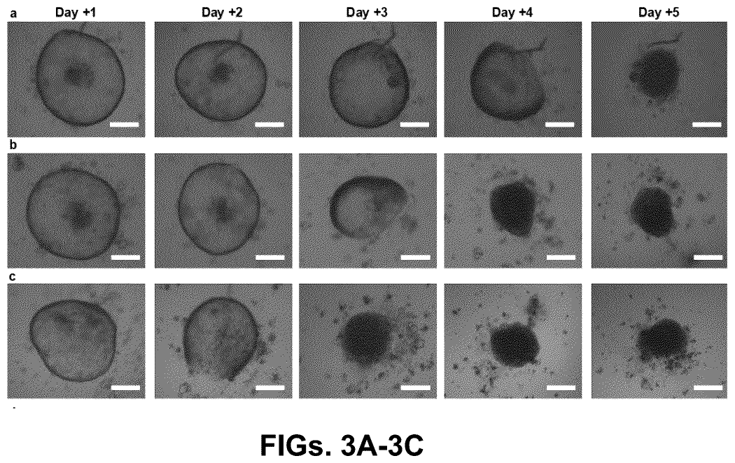

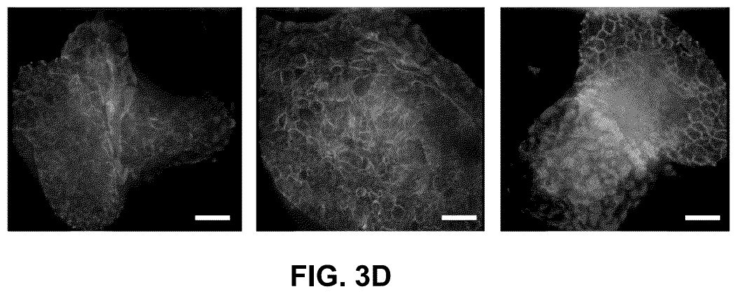

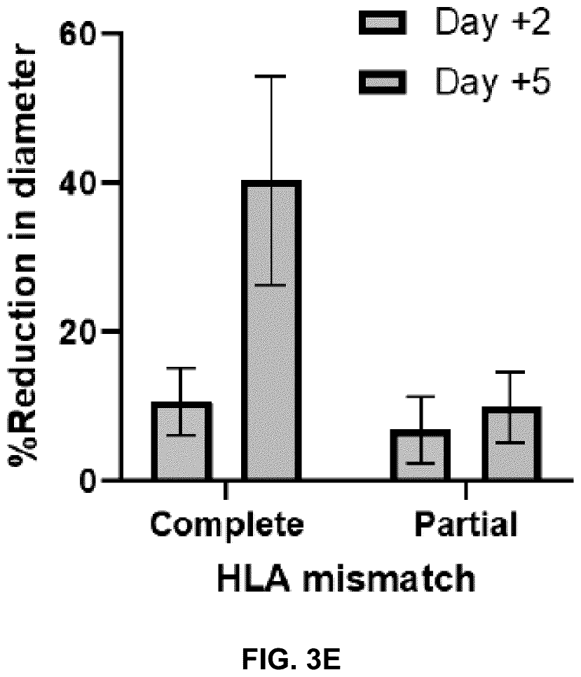

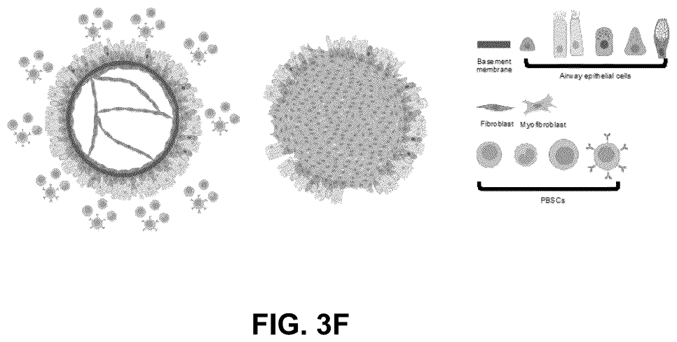

As described herein, the three-dimensional structure 100 has a diameter, and that diameter is capable of being altered by an alloimmune reaction. Such an alloimmune reaction can disrupt the plurality of cells of the second cell type (e.g., epithelial cells) and lead to contraction of the plurality of cells of the second cell type (e.g., epithelial cells) and may likewise lead to extrusion of the plurality of cells of the first cell type (e.g., fibroblasts). For example, FIG. 3F shows a schematic of the co-culture of one example described herein, where inner fibroblasts and outer epithelia making up the three-dimensional structure are surrounded by peripheral blood stem cells (left), and the subsequent immune mediated epithelial injury with fibroblast proliferation and extrusion into the epithelial defects is likewise shown.

In any of the embodiments disclosed herein, when the diameter of the three-dimensional structure is altered, the diameter may be measurably reduced thereby resulting in a second, reduced, diameter. In any of the embodiments, when the diameter is altered, the diameter may be reduced by between about 10% and about 60% resulting in a second diameter. For example, the diameter may be reduced by between about 10% and about 50%, by between about 10% and about 40%, by between about 10% and about 30%, by between about 10% and about 20%, by between about 10% and about 15%, by between about 20% and about 50%, by between about 30% and about 50%, by between about 40% and about 50%, or any amount therebetween. In one embodiment, the diameter is reduced by about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 44%, about 45%, about 46%, about 47%, about 48%, about 49%, or about 50%. In one embodiment, the reduction in diameter of the three-dimensional structure results in a decrease in roundness of the three-dimensional structure as compared to the original diameter of the three-dimensional structure, thereby resulting in a second diameter that is smaller than the original diameter of the three-dimensional structure.

The cells of the second cell type (e.g., epithelial cells) can function similar to native epithelial cells and provide secretion, selective absorption, protection, transcellular transport, or sensing functions to the three-dimensional structure 100. As would be appreciated, epithelium lines the inner surfaces of cavities in internal organs. In the three-dimensional structure described herein, epithelium can line the outer surface of the three-dimensional structure 100 and imitate an inverted organ. Accordingly, the three-dimensional structure may mimic lungs, mammary glands, kidneys, bladders, bronchial airways, tracheal airways, cornea, prostates, urethras, vaginas, cervixes, intestines, or combinations thereof. In general, E-cadherin is expressed by some epithelial cells (i.e., MCF10A and other mammary epithelial cells, lung epithelial cells, kidney epithelial cells and many other epithelial cells) and can cause regions of mainly one cell type to form. Further, E-cadherin immunostaining can be used to reveal certain epithelial cells and verify the three-dimensional structure organization. In one example, the exterior layer 106 with cells of the second cell type can interact with the external environment of the three-dimensional structure 100. In some embodiments, second cell type cells can include epithelial cells with apical markers facing outward. As such, the cells can present certain apical cell markers and transmembrane receptor cell markers along the plurality of cells of the first cell type and/or the plurality of cells of the second cell type. As would be appreciated, apical cell markers are dependent on the type of cell.

As such, the cells and cell membranes described herein can present certain cell markers and transmembrane receptor cell markers. As would be appreciated, the receptors and cell markers may be dependent on the first cell type of the first plurality of cells and/or on the second cell type of second plurality of cells. In certain embodiments, the plurality of cells of the second cell type can express certain apical cell markers including, genes relative to bronchial epithelial cells (e.g., FNI, COLIA2, MMP9, CHI3L1, LTF, ANPEP, VCAN, COLIAI, CORO6, SLCO2A1, PB, COL6A1, CYPlA1, PGGHG, COL4A4, IL34, SULTIBI, CSPG4, GPNMB, RIPOR3, ATPIOB, MUC16, SLC26A4, SERPINGI, CASC15, RSPH4A, VNN3, SPNS2, ATPIOA, MCF2L, B3GALT5, DNAil, ATP2A3, MMP3, BMF, TAGLN, GSNASI, FOXJI, LINC02015, SLC25A25-AS1, C12ort74, TPPP3, COL9A2, SLC15Al, CAPN13, CHLI, ALOX5, POU2F2, KMO, NIDI, CFTR, CFAP221, SRGAP3-AS2, ACHE, INPP5J, LINC00894, NFATC4, MYH14, VIM, MTRNR2L10, ATG9B, CUX2, PNLIPRP3, FCGBP, CCDC78, SHC2, TRO, COL4A3, VWA3B, CARD9, TENM2, ATF3, TTC25, UCKLI-ASI, CCDCl7, RGL4, SORCS2, HLA-DRA, FAM20A, NEATI, KRT75, PCDHAI 1, RPI, ZBTB46, CCDCl46, CPNE4, KCNIP3, MUC4, ZMYNDIO, NEKIO, DTHDI, LRRCIOB, PLEKHSI, MT-ND5, CFAP157, NPTXR, CYPIBI, CIS, BYES, HLA-DRBI). In certain embodiments, the apical cell markers may include markers for other genes (e.g., UBE2C, CDKN3, CEP55, MYBL2, PBK, RRM2, KIF4A, BIRC5, HJURP, ANXAIO, CENPA, HAS2, CALB2, SPC24, AURKB, GINS2, CA9, CDC45, MKI67, CKAP2L, HMMR, SHCBPI, CDC25C, DLGAP5, NCAPG, NDC80, STCI, NUSAPI, NEIL3, DEPDCI, CDC20, KIF20A, SPC25, CCNA2, PRSS3, LINC02742, TOP2A, TKI, ESCO2, ASFIB, NCAPH, BUBI, BUBIB, CLSPN, CDCA7, IGFBP6, NUF2, CDCA2, PCLAF, GTSEI, BMP7, KIF14, CCNB2, CDKI, EXOI, ILIRLI, CEMIP, FAM72A, SGOI, KNLI, SKAI, PIMREG, LMNBI, FAM72B, SNX31, OIP5, CDC6, TPX2, MNDI, CENPM, AL732437.2, ABCG2, LNCAROD, ASPM, CCNBI, SKA3, CCNE2, CNTNI, AKRIBIO, FAM72C, PRRI 1, HISTIHIA, PSRCI, SCAT8, DEPDCIB, TTK, MAD2L1, KIF15, TROAP, ARHGAPI IA, POLE2, NR5A2, RAD54L, RAD51AP1, ANLN, KIF18A, NDUFA4L2, PALDI, PTTGI, KIF2C).

Another aspect of the present disclosure provides a method of modeling Bronchiolitis Obliterans Syndrome (BOS). The method includes providing the three-dimensional structure described herein, and exposing the exterior layer of the three-dimensional structure to a plurality of blood cells, under conditions effective to model Bronchiolitis Obliterans Syndrome.

The three-dimensional structure of the present aspect includes the inverted co-culture as described herein, including an interior layer comprising a plurality of cells of a first cell type; an opposing exterior layer comprising a plurality cells of a second cell type; and a basement membrane matrix positioned between the interior layer and the exterior layer, in accordance with the previously described aspect. The three-dimensional structure of the present aspect further includes the three-dimensional structure having a diameter, where the diameter is capable of being altered by an alloimmune reaction that disrupts the plurality of cells of the second cell type and leads to contraction of the plurality of cells of the second cell type in accordance with the previously described aspect.

Bronchiolitis Obliterans Syndrome (BOS) as described herein refers to a specific and serious complication that occurs after a lung transplantation, which is characterized by lung allograft dysfunction and leads to progressive airflow obstruction and scarring of bronchioles. BOS as described herein may further refer to a manifestation of chronic pulmonary graft-versus-host disease (GVHD) and deadly complication of allogeneic hematopoietic cell transplantation (HCT). See Schwarer et al., “A Chronic Pulmonary Syndrome Associated With Graft-versus-host Disease After Allogeneic Marrow Transplantation,” Transplantation 54(6):1002-1008 (1992) and Yanik and Cooke, “The Lung as a Target Organ of Graft-Versus-Host Disease,” Seminars in Hematology 43:42-52 (2006), both of which are hereby incorporated by reference in their entirety.

In any of the embodiments disclosed herein, the plurality of blood cells comprises one or more of hematopoietic stem cells, hematopoietic circulating cells, or other leukocytes, peripheral blood stem cells (PBSCs), patient-derived cells, engineered cells, peripheral blood mononuclear cells (PBMCs), isolated lymphocytes, chimeric antigen receptor (CAR)-T cells, neutrophils, or monocytes.

The method of the present aspect may, in various embodiments, be carried out in a sample taken from a subject after a lung transplant or after a bone marrow transplant. In any of the embodiments disclosed herein, the method is carried out in vitro.

As described herein, a “sample,” “biological sample,” “test sample,” “specimen,” “sample from a subject,” and “patient sample” as used herein may be used interchangeably and may be a sample of blood, such as whole blood, tissue, skin, urine, serum, plasma, saliva, amniotic fluid, cerebrospinal fluid, placental cells or tissue, endothelial cells, leukocytes, or monocytes. The sample may be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

In any of the embodiments disclosed herein, the method further includes providing one or more healthy lung cells and one or more healthy donor HLA-mismatched immune cells. The HLA matching between the second cell type of cells and the first type of cells may be adjusted, and a closer resemblance to the clinical condition may, in various embodiments, be achieved, as evidenced for example by injury to the second cell type (e.g., epithelial cells) expansion of the first cell type (e.g., fibroblasts), and extrusion of the first cell type (e.g., fibroblasts) which may be observed through immunofluorescence analysis.

In any of the embodiments disclosed herein, the method further includes identifying one or more of a plurality of disease progression phases. In particular, the disease progression phases of BOS may be detected. A disease progression phase as described herein may include any identifiable stage of a disease which may, in various embodiments, be before detection of a disease, at an early stage of a disease, during a latent stage of a disease, during an acute stage of a disease, after detection of a disease, before, during or after treatment of a disease, after cure of a disease, or any time therebetween.

In any of the embodiments disclosed herein, the method further includes testing a treatment during at least one of the one or more of the plurality of disease progression phases. In any of the embodiments disclosed herein, the treatment is selected from one or more of a cytokine blockade, one or more of a specific cell population blockade, one or more of an agent to aid in epithelia repair, or any combination thereof. Other treatments that may be tested in accordance with the present aspect include immunosuppressive therapy, corticosteroids, antibiotics and antifungal medications, bronchodilators, macrolide antibiotics, pulmonary rehabilitation, oxygen therapy, a further lung transplantation, one or more lifestyle adjustments, or any combination thereof.

Another aspect of the present disclosure provides a method for assessing presence of or risk of developing a physiological condition. The method includes providing the three-dimensional structure described herein, and exposing the exterior layer of the three-dimensional structure to a plurality of blood cells, under conditions effective to assess presence of or risk of developing a physiological condition.

The three-dimensional structure of the present aspect includes the inverted co-culture as described herein, including an interior layer comprising a plurality of cells of a first cell type; an opposing exterior layer comprising a plurality cells of a second cell type; and a basement membrane matrix positioned between the interior layer and the exterior layer, in accordance with the previously described aspect. The three-dimensional structure of the present aspect further includes the three-dimensional structure having a diameter, where the diameter is capable of being altered by an alloimmune reaction that disrupts the plurality of cells of the second cell type and leads to contraction of the plurality of cells of the second cell type in accordance with the previously described aspect.

In any of the embodiments disclosed herein, the plurality of blood cells comprises one or more of hematopoietic stem cells, hematopoietic circulating cells, or other leukocytes, peripheral blood stem cells (PBSCs), patient-derived cells, engineered cells, peripheral blood mononuclear cells (PBMCs), isolated lymphocytes, chimeric antigen receptor (CAR)-T cells, neutrophils, or monocytes. The plurality of blood cells are present in the amount of between about 300 to about 300,000 cells. For example, the plurality of blood cells may be present in an amount of about 300 cells, about 400 cells, about 500 cells, about 600 cells, about 700 cells, about 800 cells, about 900 cells, about 1,000 cells, about 1,500 cells, about 2,000 cells, about 2,500 cells, about 3,000 cells, about 3,500 cells, about 4,000 cells, about 4,500 cells, about 5,000 cells, about 5,500 cells, about 6,000 cells, about 6,500 cells, about 7,000 cells, about 7,500 cells, about 8,000 cells, about 8,500 cells, about 9,000 cells, about 9,500 cells, about 10,000 cells, about 15,000 cells, about 20,000 cells, about 50,000 cells, about 100,000 cells, about 150,000, about 200,000 cells, about 250,000 cells, or about 300,000 cells. In various examples, the plurality of blood cells may be present in an amount of between about 300 to about 250,000 cells, between about 300 to about 200,000 cells, between about 300 to about 150,000 cells, between about 300 to about 100,000 cells, between about 300 to about 50,000 cells, between about 300 to about 40,000 cells, between about 300 to about 30,000 cells, between about 300 to about 20,000 cells, between about 300 to about 10,000, or any amount therebetween.

In any of the embodiments disclosed herein, the physiological condition may include a recapitulation of one or more diseases involving an immune response. The physiological condition may, in one embodiment, include one or more diseases involving epithelial-stromal-blood cells.

The method of the present aspect may, in various embodiments, further include varying degrees of human leukocyte matching of the three-dimensional structure for at least one of the cells of the first cell type, the second cell type, and/or the blood cells.

In any of the embodiments disclosed herein, the varying degrees of human leukocyte matching for at least one of the cells of the three-dimensional structure includes varying the degree of antigen matching.

In any of the embodiments disclosed herein, the varying degrees of human leukocyte matching for at least one of the cells of the three-dimensional structure includes varying the degree of protein level.

In any of the embodiments disclosed herein, the varying degrees of human leukocyte matching includes fully matching human leukocyte antigens (HLA) between one or more cells of the second cell type and one or more blood cells. In any of the embodiments disclosed herein, the varying degrees of matching includes tuning human leukocyte antigens (HLA) mismatch between one or more cells of the second cell type and one or more blood cells.

In any of the embodiments disclosed herein, the method further includes culturing a plurality of the three-dimensional structure in a plurality of wells in a single-organoid-per-well format; and manipulating a growth factor level in the plurality of wells.

In any of the embodiments disclosed herein, the method further comprises isolating RNA from the single three-dimensional structure. In any of the embodiments disclosed herein, the method further includes sequencing RNA isolated from the single three-dimensional structure. In any of the embodiments disclosed herein, the method further includes harvesting a supernatant from the plurality of wells and assaying the supernatant.

Another aspect of the present disclosure provides a method of identifying one or more biomarkers of a disease. The method includes providing the three-dimensional structure described herein; exposing the exterior layer of the three-dimensional structure to a plurality of blood cells; and identifying one or more biomarkers of a disease.

The three-dimensional structure of the present aspect includes the inverted co-culture as described herein, including an interior layer comprising a plurality of cells of a first cell type; an opposing exterior layer comprising a plurality cells of a second cell type; and a basement membrane matrix positioned between the interior layer and the exterior layer, in accordance with the previously described aspect. The three-dimensional structure of the present aspect further includes the three-dimensional structure having a diameter, where the diameter is capable of being altered by an alloimmune reaction that disrupts the plurality of cells of the second cell type and leads to contraction of the plurality of cells of the second cell type in accordance with the previously described aspect.

In any of the embodiments disclosed herein, the method of the present aspect further includes diagnosing or prognosing a disease based on the presence or absence of one or more biomarkers of disease.

Diagnosing as described herein includes detecting the presence of a disease. Diagnosing as described herein further includes the process of identifying a disease by its signs, symptoms and/or results of one or more tests, such as those provided herein. The conclusion reached through that process is referred to a diagnosis. Forms of testing commonly performed include blood tests, medical imaging, genetic analysis, urinalysis, biopsy and analysis of biological samples obtained from a subject. In one example, diagnosis is determining whether a subject has BOS.

Prognosing as described herein includes determining the susceptibility to developing a disease. Prognosis as described herein further includes the determination of whether a subject will develop a disease in the future, such as the predisposition of a subject to develop BOS in the future.

In any of the embodiments disclosed herein, the biomarkers of disease may be, but are not limited to, interleukin-6 (IL-6), c-reactive protein (CRP), tumor necrosis factor-alpha (TNF-α), donor-derived cell-free DNA (dd-cfDNA), lymphocyte count, neutrophil count, surfactant proteins (SP-A, SP-D), krebs von den Lungen-6 (KL-6), matrix metalloproteinases (MMP), transforming growth factor-beta (TGF-0), procalcitonin (PCT), galactomannan, cytomegalovirus (CMV) DNA, pathogen-specific PCR, b-type natriuretic peptide (BNP), lactate dehydrogenase (LDH), total protein, albumin, erythrocyte sedimentation rate (ESR), fibrinogen, or any combination thereof.

In any of the embodiments disclosed herein, the method further includes identifying targetable pathways for treatment of the disease. In any of the embodiments disclosed herein, the targetable pathway for treatment of the disease may include a mechanistic target of rapamycin (mTOR) pathway in T cells.

In any of the embodiments disclosed herein, the method of the present aspect may further include testing a response of the one or more biomarkers of a disease (e.g., BOS) in response to one or more interventions. The one or more interventions, in various embodiments, may include, but are not limited to, a TNF-alpha blockade.

Suitable biological samples in accordance with the present disclosure include biological samples such as blood, blood serum, blood plasma, cerebrospinal fluid, urine, saliva, tissue. In any of the embodiments disclosed herein, the biological sample is selected from the group consisting of blood, serum, plasma, urine, saliva, tears, mucus, lymph, interstitial fluid, cerebrospinal fluid, pus, breast milk, amniotic fluid, or any combination thereof.

The phrase “derived from” as used herein includes cells or a biological sample and indicates that the cells or the biological sample were obtained from the stated source at some point in time. For example, a cell derived from a subject can include a blood cell obtained directly from the subject (e.g., unmodified). In some instances, a cell derived from a given source undergoes one or more rounds of cell division and/or cell differentiation such that the original cell no longer exists, but the continuing cell (e.g., daughter cells from all generations) will be understood to be derived from the same source. The term includes directly obtained from, isolated and cultured, or obtained, frozen, and thawed. The term “derived from” may also refer to a component or fragment of a cell obtained from a tissue or cell, including, but not limited to, a protein, a nucleic acid, a membrane or fragment of a membrane, and the like.

Another aspect of the present disclosure provides a method of making a three-dimensional structure. The method includes providing a plurality of cells of a first cell type; providing a plurality cells of a second cell type; providing a basement membrane matrix material; and co-culturing the plurality of cells of the first type, the plurality of cells of the second cell type, and the basement membrane matrix material under conditions effective to form the three-dimensional structure described herein.

The three-dimensional structure of the present aspect includes the inverted co-culture as described herein, including an interior layer comprising a plurality of cells of a first cell type; an opposing exterior layer comprising a plurality cells of a second cell type; and a basement membrane matrix positioned between the interior layer and the exterior layer, in accordance with the previously described aspect. The three-dimensional structure of the present aspect further includes the three-dimensional structure having a diameter, where the diameter is capable of being altered by an alloimmune reaction that disrupts the plurality of cells of the second cell type and leads to contraction of the plurality of cells of the second cell type in accordance with the previously described aspect.

The method may include mixing an extracellular matrix mixture at a first temperature with a culture medium at a second temperature, the second temperature greater than the first temperature. This step can include heating the culture medium to a temperature greater, or warmer, than the temperature of the extracellular matrix mixture. Additionally, or alternatively to, this step can include cooling the extracellular matrix mixture to a temperature lower than, or cooler, than the temperature of the culture medium. The culture medium temperature can range from about 20° C. to about 40° C. (e.g., from about 21° C. to about 40° C., from about 22° C. to about 40° C., from about 23° C. to about 40° C., from about 24° C. to about 40° C., from about 25° C. to about 40° C., from about 26° C. to about 40° C., from about 27° C. to about 40° C., from about 28° C. to about 40° C., from about 29° C. to about 40° C., from about 30° C. to about 40° C., from about 31° C. to about 40° C., from about 32° C. to about 40° C., from about 33° C. to about 40° C., from about 34° C. to about 40° C., from about 35° C. to about 40° C., from about 36° C. to about 40° C., from about 37° C. to about 40° C., from about 38° C. to about 40° C., from about 39° C. to about 40° C., and any additional ranges and integers not expressly stated, e.g., 27.5° C. to about 37.2° C.). In general, a culture medium temperature greater than or equal to room temperature is preferred. As would be appreciated by those of skill in the relevant art, protocols for formation of apical-in organoids require mixing extracellular matrix mixtures with a chilled culture medium, or a culture medium at a temperature lower than the extracellular matrix mixture and at higher extracellular matrix concentrations. In contrast, formation of the three-dimensional structures described herein may include mixing extracellular matrix mixtures with a warmed culture medium, or a culture medium at a temperature higher than the extracellular matrix mixture and at lower extracellular matrix concentrations that is typical as described in manufacturer instructions. More generally, an extracellular matrix mixture may be added to warm medium in small amounts where the conventional method would simply dilute the mixture and preclude gel formation. In the method described herein, the small amount of extracellular matrix mixture may be added to medium at a temperature above a lower critical solution temperature of the extracellular matrix mixture. A lower critical solution temperature is a critical temperature at which the components of a mixture are miscible for all compositions, or not gelled.

In some embodiments, prior to mixing the extracellular matrix mixture, a stock solution of extracellular matrix mixture can be prepared. Highly concentrated extracellular matrix mixtures may form a gel-like consistency at concentrations above conventional recommended concentrations. For instance, a concentration above 3 mg/mL of extracellular matrix mixture may result in gelling. In certain embodiments, extracellular matrix mixture may gel and form a minimal scaffold in which the three-dimensional structure can be formulated. In some embodiments, a concentration of extracellular matrix mixture above about 4 mg/mL may be used to form the three-dimensional structure (e.g., above about 4.5 mg/mL, above about 5 mg/mL, above about 5.5 mg/mL, above about 6 mg/mL, above about 6.5 mg/mL, above about 7 mg/mL, above about 7.5 mg/mL, above about 8 mg/mL, above about 8.5 mg/mL, above about 9 mg/mL, above about 9.5 mg/mL, above about 10 mg/mL, above about 10.5 mg/mL, above about 11 mg/mL, above about 11.5 mg/mL, above about 12 mg/mL, and any additional concentration ranges and integers of such not expressly stated, e.g., from about 7.4 mg/mL to about 10.7 mg/mL).

In some embodiments, the method may include mixing less than 1 mg/mL of the extracellular matrix mixture with the culture medium to form the concentrations described above and promote gelling (e.g., less than 0.95 mg/mL, less than 0.90 mg/mL, less than 0.85 mg/mL, less than 0.80 mg/mL, less than 0.75 mg/mL, less than 0.70 mg/mL, less than 0.65 mg/mL, less than 0.60 mg/mL, less than 0.55 mg/mL, less than 0.50 mg/mL, less than 0.45 mg/mL, less than 0.40 mg/mL, less than 0.35 mg/mL, less than 0.30 mg/mL, less than 0.25 mg/mL, less than 0.20 mg/mL, less than 0.15 mg/mL, less than 0.10 mg/mL, less than 0.08 pg/mL, less than 0.06 mg/mL, less than 0.04 mg/mL, less than 0.02 mg/mL, less than 0.01 mg/mL, and any additional range and integers of such not expressly stated, e.g., less than 0.68 mg/mL). Additionally, or alternatively thereto, the method may include mixing less than 500 μg/mL of the extracellular matrix mixture with the culture medium (e.g., less than 480 μg/mL, less than 440 μg/mL, less than 400 μg/mL, less than 360 μg/mL, less than 320 μg/mL, less than 280 μg/mL, less than 240 μg/mL, less than 200 μg/mL, less than 160 μg/mL, less than 120 μg/mL, less than 90 μg/mL, less than 80 μg/mL, less than 70 μg/mL, less than 60 μg/mL, less than 50 μg/mL, less than 40 μg/mL, less than 30 μg/mL, less than 20 μg/mL, less than 10 μg/mL, and any additional range and integers of such not expressly stated, e.g., less than 452 μg/mL).

In some embodiments, adding too much or too little of the extracellular matrix can preclude formation of the three-dimensional structure. For instance, adding too much extracellular matrix can result in multiple small structure, or, adding too little extracellular matrix can form small, dense cells, not three-dimensional structures. Generally, the total volume of gel formed is similar to the size of the three-dimensional structure to be formed.

Once at least the extracellular matrix mixture is mixed with a culture medium, the method can include culturing a plurality of cells of a first cell type and/or a plurality of cells of a second cell type in the extracellular matrix mixture and culture medium. The method may include culturing the one or more plurality of cells and the extracellular matrix mixture and culture medium in a non-stick surface culture system, such as, for example, one or more of a hanging drop system, an ultra-low attachment system, a hydrogel well system, an ultrasound levitation system, or any suitable research and industrialized methods used for making three-dimensional structures or organoids. In some embodiments, the method may include culturing the plurality of cells of the second cell type and the plurality of cells of the first type simultaneously. Additionally or alternatively thereto, the method can be carried out by culturing the plurality of cells of the first cell type before culturing the plurality of cells of the second cell type and/or the plurality of cells of the third cell type.

The method may further include forming the three-dimensional structure having an interior chamber enclosed by the plurality of cells of the second cell type configured to interface with an environment external to the three-dimensional structure. The method can further include entrapping the extracellular matrix mixture within the interior chamber of the three-dimensional structure. The method may further include forming the interior layer with a plurality of cells of the first cell type configured to interface with the interior chamber, the basement membrane matrix, and the exterior layer of the three-dimensional structure. In some embodiments, the method may also include forming a tissue layer between the first plurality of cells and the interior chamber of the three-dimensional structure. As would be appreciated by those of skill in the art, the three-dimensional structure described herein may mimic complex organs and form additional three-dimensional structures within, adjacent to, and/or outside the internal chamber of the three-dimensional structure. Additionally, or alternatively thereto, the three-dimensional structure may have folds or crypt-like domains such that only a portion of the plurality of cells of the second cell type is interfacing with the environment external to the three-dimensional structure. In some embodiments, forming the three-dimensional structure of the method described herein can result in from about 5% to about 100% of the plurality of cells of the second cell type interfacing with the environment external to the three-dimensional structure (e.g., from about 5% to about 100%, from about 10% to about 100%, from about 15% to about 100%, from about 20% to about 100%, from about 25% to about 100%, from about 30% to about 100%, from about 35% to about 100%, from about 40% to about 100%, from about 45% to about 100%, from about 50% to about 100%, from about 55% to about 100%, from about 60% to about 100%, from about 65% to about 100%, from about 70% to about 100%, from about 75% to about 100%, from about 80% to about 100%, from about 85% to about 100%, from about 90% to about 100%, from about 95% to about 100%, from about 96% to about 100%, from about 97% to about 100%, from about 98% to about 100%, from about 99% to about 100%, from about 99.5% to about 100%, from about 99.9% to about 100%, from about 99.99% to about 100%, and any additional range and integers of such not expressly stated).

The method can further include exposing the environment external to the three-dimensional structure to a virus, a bacteria, a fungi, a cancer cell, an immune cell, a stem cell, a drug, or combinations thereof. As would be appreciated, exposing epithelial cells positioned on the external surface of a three-dimensional structure can be achieved by adding the virus, bacteria, fungus, cancer cell, immune cell, stem cell, or drug to the environment (e.g., medium, scaffold, or other) in which the three-dimensional structure is in.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

### EXAMPLES

The following Examples are presented to illustrate various aspects of the present application, but are not intended to limit the scope of the claimed application.

### Materials and Methods

2D human primary bronchial epithelial basal cell expansion—Primary normal human tracheal/bronchial epithelial cells, hTBECs, (Lonza, CC-2540S) were expanded in Pneumacult™-Ex Plus basal medium (STEMCELL Technologies, #05041) supplemented with Pneumacult™-Ex Plus 50× supplement (STEMCELL Technologies, #05042) and 0.5 mL of 200 mM hydrocortisone using the manufacturer's recommended seeding density of 3,500 cells/cm² on a T-75 cell culture flask (Corning). The cells were subcultured at the confluence of 70-80%. Upon collection, cells were passaged, used for organoid culture, or cryopreserved. Cells beyond passage number 4 were not used, as they did not yield adequate organoid formation.

Preparation of hanging-drop plates—For at least one day prior to organoid seeding, a 384-well hanging-drop (HD) plate custom designed with injection moulding (Xcentric) was soaked in 0.1% solution of Pluronic F108 (Sigma-Aldrich, 542342) in distilled water (diH₂O) (Gibco, 15230). On the day of organoid seeding, the HD plate was rinsed off with diH₂O and then air dried. The plate was then UV-sterilized (Analytik Jena, CL-1000) for 20 minutes on each side. While waiting, the wells of a sterile non-tissue-treated round-bottom 96-well plate (Corning, 351177) were each filled with 150 μl of diH₂O supplemented with 1% pen-strep. When the HD plate was fully sterilized, it was placed (sandwiched) between the bottom and the lid of the 96-well plate. The two troughs of the short-edged sides were loaded with diH₂O, which were then stuffed with sterile gauze pads. Lastly, the long edges were each added with ˜1 ml of diH₂O.

Hanging-drop AORB culture—The seeding solution was composed of Pneumacult™ Airway Organoid Basal Medium (Stemcell Technologies, #05061) supplemented with the following additives: Pneumacult™ Airway Organoid Seeding Supplement (Stemcell Technologies, #05062), methylcellulose (Sigma-Aldrich, 94378), and growth factor reduced Matrigel (Corning, 356231). First, the seeding supplement was added to the base medium (10% concentration). Next, an appropriate volume of methylcellulose was added (0.24% final concentration at the target volume) and the solution warmed in a 37° C. bead bath (Fisher Scientific, GSGPD10). Once warm, Matrigel (at 5.35 μg/ml to 16.05 μg/ml final concentration) was then deliberately kept cold in ice and added to the pre-warmed media. Next, the desired volume of cell suspension at a final concentration of 120,000 cells/ml (3000 cells per 25 μl of the droplet) was added. Throughout the process, it was crucial to mix components very thoroughly. Immediately after the seeding solution was prepared, an eight-channel repeater pipette (Eppendorf, 4861000120) was used to load the HD plate in a staggered zigzag pattern (8 alternating rows×24 columns=192 wells). This pattern minimized adjacent wells with droplets from merging.

Maintenance of AORBs—The organoids were monitored as needed with a high-throughput imaging system (Thermo Scientific, EVOS FL Auto 2). At days 3-4 of organoid seeding and every three days thereafter, the media exchange procedure was performed using a liquid handler robot (Analytik Jena, CyBio FeliX). Volumes to be removed from and added to the organoids were programmatically controlled using the CyBio Composer software. The organoid differentiation medium (ODM) to be exchanged with was Pneumacult™ Airway Organoid Basal Medium (Stemcell Technologies, #05061) supplemented with Differentiation Supplements (Stemcell Technologies, #05063). It may be necessary to pre-fill wells around the outer edges with an additional medium (typically no more than ˜5 μl) to prevent aspiration of organoids. Moreover, it is advised to limit the time of handling HD plates outside the humidity-controlled incubator as much as possible to minimize evaporation of the liquid drops. The HD plate was kept in a cell culture incubator (Thermo Scientific, VIOS 160i) with 5% CO₂ injection at 37° C.

2D expansion of fibroblasts—Normal human lung fibroblast (NHLF) cells (Lonza, CC-2512) isolated from the same donor as the 2^{nd} NHBE donor were expanded in FGM™ fibroblast growth medium (Lonza, CC-3132). IMR90 cells (ATCC, CCL-186) were expanded in Dulbecco's Modified Eagle's Medium (DMEM) (ATCC, 30-2002) supplemented with 10% FBS (Gibco, A52560801) and 100 U/mL penicillin/streptomycin (pen/strep) (Gibco, 15140122). No antibiotics were used for the primary cells. The cells were subcultured at a confluence of 70-80%. Upon collection, cells were passaged, used for organoid seeding, or cryopreserved. For NHLF, cells beyond passage number 5 were not used, whereas IMR90 cells were used at passage number <10.

Epithelial-fibroblast co-culture organoids—The compositions of organoid seeding and differentiation media were identical to those described above. The only difference lay in the ratios of epithelial-to-fibroblast seeding cell numbers, optimized based on three conditions (6:1, 3:1, 2:1), with the number of epithelial cells fixed at 3000. The amount of Matrigel/drop was also optimized from four conditions (˜1350, 2000, 2700, and 4000 ng).

Immunofluorescence assay—The whole-mount IF and imaging protocol is as outlined in Lee et al., “Development of Robust Antiviral Assays Using Relevant Apical-Out Human Airway Organoids,” bioRxiv 12:2024 (2024), which is hereby incorporated by reference in its entirety, with modifications, for example, in some of the antibodies and stains used. The antibodies used as markers for epithelial cells and fibroblasts were E-cadherin (Invitrogen, 13-1700) and vimentin (Invitrogen, MA5-14564), respectively. For real-time cell tracking via fluorescence, PBSCs were stained with NucBlue (Invitrogen, R37605) prior to introduction to co-culture organoids, while fibroblasts were stained with NucRed (Invitrogen, R37106) before organoid co-seeding.

PBSC isolation—In brief, two healthy stem cell donors were administered 10 μg/kg of granulocyte-colony stimulating factor (G-CSF) subcutaneously for 5 days to induce hematopoietic proliferation, followed by leukapheresis for clinical purposes per institutional protocol. Samples had been allocated to research with consent.

Introducing PBSCs to co-culture organoids—Cryopreserved PBSCs were thawed and diluted appropriately in human serum derived from male AB plasma (MilliporeSigma, H4522) at three different doses (300, 3000, and 30000 per organoid). A liquid handler robot (Analytik Jena, CyBio FeliX) was used to dispense the PBSC suspension into individual drops containing organoids.

Supernatant collection—Supernatants were collected from the first wash during each media change routine. Briefly, the liquid handler robot removes a pre-determined volume of supernatant into a collection plate, after which samples were pooled into a tube, frozen, and stored until further assay was performed.

### Example 1—Minimal Matrigel Scaffolding Facilitates Successful Formation of Epithelial-Fibroblast Co-Culture Organoids

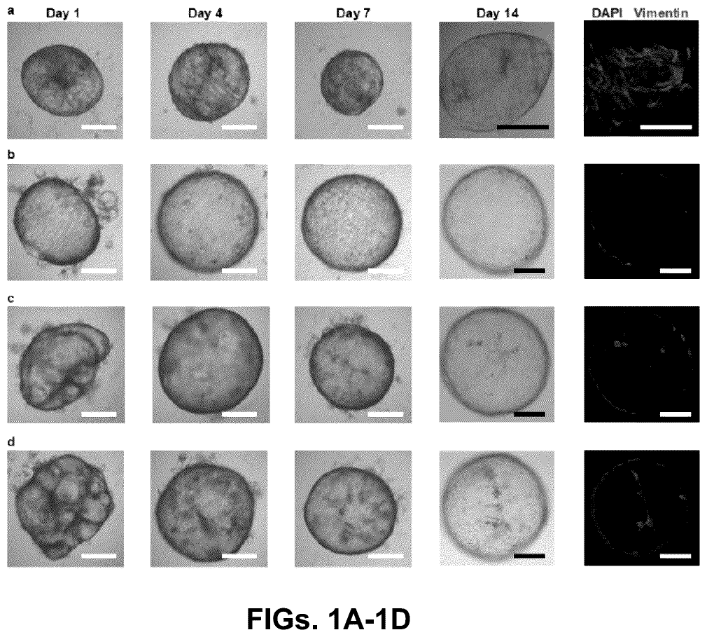

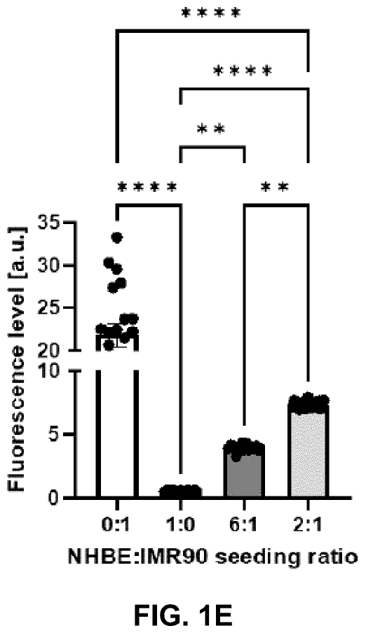

The previously described minimal Matrigel scaffolding method (Parigoris et al., “Cancer Cell Invasion of Mammary Organoids With Basal-in Phenotype,” Advanced Healthcare Materials 10(4):e2000810 (2021); Parigoris et al., “Extended Longevity Geometrically Inverted Proximal Tubule Organoids,” Biomaterials 290:121828 (2022); U.S. Patent Application Publication No. 2023/0194506A1 to Parigoris et al.; Lee et al., “Development of Robust Antiviral Assays Using Relevant Apical-Out Human Airway Organoids,” bioRxiv 12:2024 (2024); Lee et al., “High Throughput Formation and Image-based Analysis of Basal-in Mammary Organoids in 384-well Plates,” Scientific Reports 12(1):317 (2022); Mertz et al., “Triple-negative Breast Cancer Cells Invade Adipocyte/preadipocyte-encapsulating Geometrically Inverted Mammary Organoids,” Integrative Biology 15:zyad004 (2023), all of which are hereby incorporated by reference in their entirety) was adapted, to successfully form inverted epithelial-fibroblast co-culture organoids (FIGS. 1A-1E and FIGS. 2A-2F). The inverted organoid permits the epithelial cell layer to be positioned outwardly on the basement membrane with an interior layer of fibroblasts, enabling direct exposure of the epithelia to PBSCs (FIG. 2A). Initially, the co-culture was optimized to determine the ideal seeding ratio between NHBE to human fibroblast cell line (IMR90) cells (FIGS. 1A-IE). Seeding with fibroblasts alone led to the formation of organoids with relatively smaller diameters and prominent vimentin-positive IF stains (FIG. 1A). There was minimal vimentin IF signal in the NHBE-only organoids as expected, indicative of little to no epithelial-to-mesenchymal transition (EMT) (FIG. 1B). Adjusting the NHBE-to-IMR90 ratio from 6:1 to 2:1 resulted in an increase in vimentin signal in the core (FIGS. 1C-1E). Consistent with prior findings (Lee et al., “Development of Robust Antiviral Assays Using Relevant Apical-Out Human Airway Organoids,” bioRxiv 12:2024 (2024), which is hereby incorporated by reference in its entirety), titrating Matrigel amount can enable robust and efficient control over organoid diameters. Moreover, the ratio of organoid-to-fibroblast medium influenced the extent of the fibroblast interior.

Upon refining the co-culture conditions based on the IMR-90 cell line, the co-culture involving NHBE and NHLF was optimized (FIGS. 2A-2F). These fibroblasts were sourced from a single donor to ensure HLA matching between the lung epithelia and fibroblasts. Advanced microscopy technique revealed an expected hollow core present in epithelial-only organoids, and fibrous networks formed by fibroblasts within the co-culture organoids (FIGS. 1A-1E). This advanced technique also supported live-cell imaging, which visualized beating cilia with noticeable increase in ciliated cells over time. Similar to the experiments with the IMR-90 cell line (FIGS. 1A-1E), increasing the ratio of NHBE-to-NHLF also resulted in increased vimentin staining (FIG. 1E). The co-seeding of 3000 NHBE and 1500 NHLF showed favorable organoid formation; therefore, this ratio was chosen for subsequent experiments.

### Example 2—Modelling BOS Using Tri-Culture Organoids

Mobilized PBSCs from a healthy HLA-mismatched adult donor was introduced onto the NHBE-IMR90 organoids with dose escalation of PBSC from 300 to 30,000 cells (FIGS. 3A-3F).

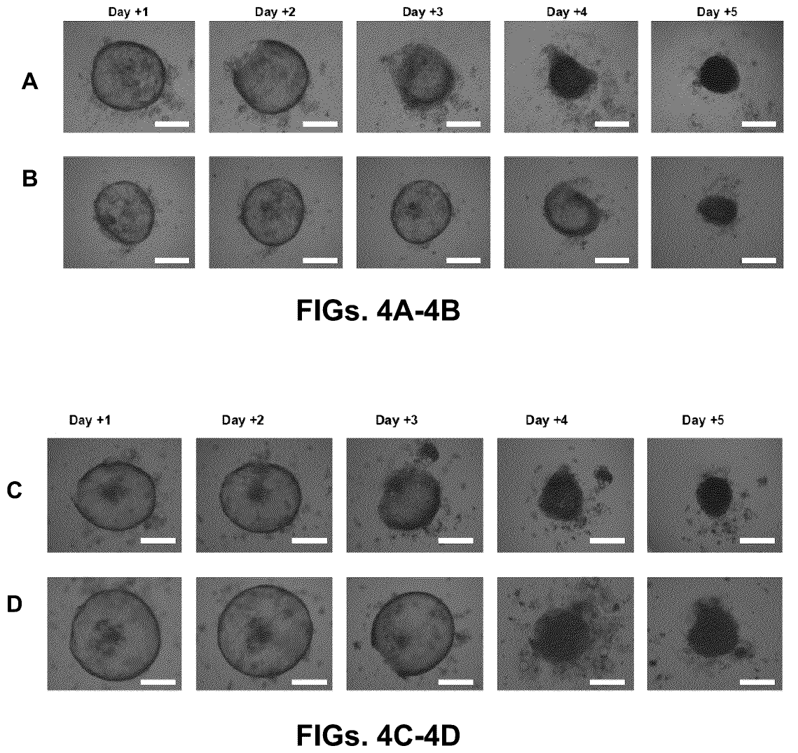

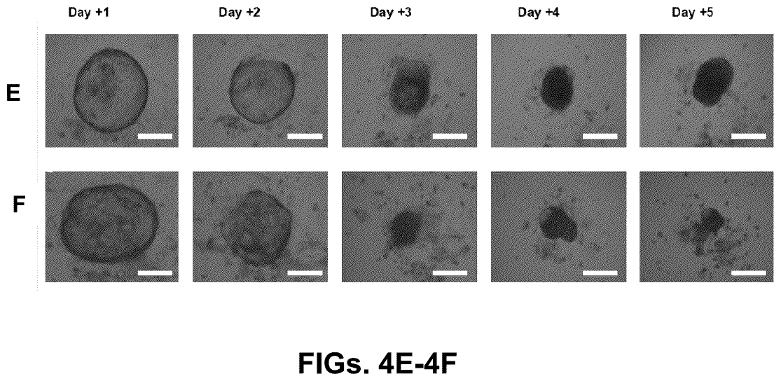

After exposure to the escalating doses of mismatched PBSCs, epithelial disruption was observed in a dose-dependent manner (FIGS. 3A-3F, FIGS. 4A-4F). More specifically, the earliest onset of breach of the epithelium occurred with the highest dose at 30,000 PBSCs, with similar damage after smaller doses occurring days later. All organoid conditions showed initial disruption of the epithelia, fibroblasts expansion that extruded to the external aspect of the organoid, followed by contraction the organoids. Reduction in organoid diameter by days 2 and 5 post-PBSC addition was quantified (FIG. 3E). By day 2, organoid sizes decreased by 10.7±4.5% and by 40.2±14.0% on day 5 after PBSC exposure. Using the NHBE-NHLF organoid, a similar pattern was observed with epithelial destruction and reduction in size with 2 HLA-mismatched donor PSBC. However, the organoids were less spherical and the timing of the epithelial breaching was more variable than that observed with the NHBE-IMR90 organoids.

### Example 3—Discussion

The pathogenesis of BOS remains poorly understood due to the absence of animal models that fully recapitulate the disease, the rarity of this manifestation of cGVHD, the inaccessible organ site of disease, the delayed diagnosis and associated the high mortality. Although murine models for BOS have been published, obliterated bronchioles remain a rare feature in these models, reported in 1.9-8.5% of the airways in 15.5-50% of the mice (Panoskaltsis-Mortari et al., “A New Murine Model for Bronchiolitis Obliterans Post-Bone Marrow Transplant,” Am. J. Respir. Crit. Care Med. 176:713-723 (2007), which is hereby incorporated by reference in its entirety), conferring poor penetrance and incompletely mimicking the clinical condition, which leads to >70% of airways with intraluminal fibrosis in humans. This is likely linked differences in the cell populations between murine and human bronchioles. Murine bronchiole epithelia lack basal cells and submucosal glands, cells crucial for repair after epithelial injury and protection from pathogens, respectively. Swatek et al., “Depletion of Airway Submucosal Glands and TP63+KRT5+ Basal Cells in Obliterative Bronchiolitis,” Am. J. Respir. Crit. Care Med. 197:1045-1057 (2018), which is hereby incorporated by reference in its entirety. In mice, these elements are confined to the trachea, where obliteration is successfully induced after toxic injury with high penetrance. Swatek et al., “Depletion of Airway Submucosal Glands and TP63+KRT5+ Basal Cells in Obliterative Bronchiolitis,” Am. J. Respir. Crit. Care Med. 197:1045-1057 (2018), which is hereby incorporated by reference in its entirety. The co-culture model described herein, comprising human primary lung cells, could overcome the limitations of murine models to more accurately mimic the clinical disease. To facilitate this, an inverted organoid model was first developed, mitigating the need for organoid disruption (with needle microinjection of PBSCs) to expose the epithelia to the mismatched PBSC product. Lee et al., “Development of Robust Antiviral Assays Using Relevant Apical-Out Human Airway Organoids,” bioRxiv 12:2024 (2024), which is hereby incorporated by reference in its entirety. Next, a key population in BOS pathogenesis was incorporated in the model, fibroblasts. This required optimization of the ratio of human epithelia to a fibroblast cell line to generate the co-culture. The model also incorporated HLA mismatched PBSCs, as this cell source has been linked to cGVHD and BOS specifically. Chien et al., “Bronchiolitis Obliterans Syndrome After Allogeneic Hematopoietic Stem Cell Transplantation—An Increasingly Recognized Manifestation of Chronic Graft-versus-Host Disease,” Biology of Blood and Marrow Transplantation 16(1 Suppl):S106-S114 (2010) and Versluys et al., “Strong Association between Respiratory Viral Infection Early after Hematopoietic Stem Cell Transplantation and the Development of Life-Threatening Acute and Chronic Alloimmune Lung Syndromes,” Biology of Blood and Marrow Transplantation 16:782-791 (2010), both of which are hereby incorporated by reference in their entirety. Finally, the organoid BOS model if the first to incorporate healthy lung cells, healthy donor HLA-mismatched immune cells, and fibroblasts, permitting study of the genesis of BOS. Dose escalation experiments were performed both to identify the optimal PBSC dose for the model and confirm the role of the PBSC in the genesis of the epithelial injury.

Given the HLA-mismatch of the fibroblast cell line, the role of HLA-matched fibroblasts to the organoid model was investigated. Thus, fibroblasts were obtained that were HLA-matched to the lung epithelia and basal cells. However, while the pattern of epithelial injury was conserved, the extent of the injury in relation to the time after exposure and dose was less consistent. Further, the organoids were no longer spherical, rather bulbous, precluding measurements of the diameter as a marker of disease progression and severity. As others have published, this is likely due to variability in the activity and function in primary fibroblasts. Rayner et al., “Optimization of Normal Human Bronchial Epithelial (NHBE) Cell 3D Cultures for in vitro Lung Model Studies,” Sci. Rep. 9:500, pp. 1-10 (2019), which is hereby incorporated by reference in its entirety. However, the same pattern of epithelial injury was observed, with breaching of the organoid surface, evidence of fibroblast expansion and extrusion to cover the injured epithelia, confirmed by immunofluorescence.

Several aspects of the BOS organoid demonstrated fidelity with the clinical condition. Biologically, BOS after HCT involves injury to the epithelial mediated by activated allo-immune cells. Studies have consistently shown that PBSC is a risk factor for BOS, a stem cell source that includes more activated T cells than the marrow or cord blood (alternative sources). Further, the fibroblasts filling of the injured epithelia mirrors the intraluminal fibrosis seen on pathology of obliterated bronchioles. While the lack of available HLA-matched peripheral blood cells to the epithelial cells precluded an autologous model to compare, the escalation in epithelial injury with increasing doses, and lack of injury in exposed organoids support that the organoid represents an allo-immune lung epithelial injury with fibroblast filling of the defects, akin to the clinical condition.

To the best of knowledge, this organoid model was the first human airway model for studying the earliest genesis of BOS, using healthy lung cells and HLA-mismatched donor PBSC. This model includes the clinically implicated cell types, including human airway basal cells, differentiated epithelia, fibroblasts, and hematopoietic cells. Its unique “inside-out” morphology, with outward-facing epithelia, facilitated facile exposure and interaction with hematopoietic cells in the milieu, enabling the recognition and attack of airway epithelia in the native microenvironment without injury to the organoid. Furthermore, the airway organoid generated from the method of Matrigel scaffolding has demonstrated efficient and faithful viral infection and successfully predicted response to viral treatments. Lee et al., “Development of Robust Antiviral Assays Using Relevant Apical-Out Human Airway Organoids,” bioRxiv 12:2024 (2024), which is hereby incorporated by reference in its entirety. Further, the data consistently generated the same pattern across different donors and lung recipient cells, and suggested that size of the organoid (diameter) may be a marker of disease, reflecting epithelial injury and fibroblast expansion.

In vitro modelling enabled the manipulation of engineering aspects, such as the varying degrees of HLA (mis)match between the epithelial-fibroblast pair (representing the recipient) and PBSCs (the allograft donor). Other advantages included single-organoid analysis and high-throughput capability, which can facilitate ease of interpretation and efficient therapeutic screening.

In summary, the BOS organoid model described herein offers an innovative, efficient, and physiologically relevant bench-side approach through tri-culture organoids composed of three cell types: airway epithelial cells, fibroblasts, and HLA-mismatched PBSC, tailored for studying BOS. As the first of its kind employing human primary cells, the model can provide a more accurate representation of the disease, thereby facilitating the discovery of key affected pathways and disease drivers. This model could elucidate the pathogenesis of BOS, identify biomarkers of disease for diagnosis or prognosis, identify targetable pathways for treatment, test novel treatments at different phases of disease progression, and testing biomarker response to interventions. These findings may thus generate highly translatable and broadly generalizable insights in BOS after HCT or lung transplant, highly morbid conditions.

Although preferred embodiments have been depicted and described in detail herein, it will be apparent to those skilled in the relevant art that various modifications, additions, substitutions, and the like can be made without departing from the spirit of the application and these are therefore considered to be within the scope of the application as defined in the claims which follow.

What is claimed:

1. A three-dimensional structure comprising:
an inverted co-culture comprising:
an interior layer comprising a plurality of cells of a first cell type;
an opposing exterior layer comprising a plurality cells of a second cell type; and
a basement membrane matrix positioned between the interior layer and the exterior layer,
wherein the three-dimensional structure has a diameter, and wherein said diameter is capable of being altered by an alloimmune reaction that disrupts the plurality of cells of the second cell type and leads to contraction of the plurality of cells of the second cell type.

2. The three-dimensional structure of claim 1, wherein the first cell type comprises stromal cells.

3. The three-dimensional structure of claim 2, wherein the stromal cells comprise fibroblasts.

4. The three-dimensional structure of claim 1, wherein the second cell type comprises epithelial cells.

5. The three-dimensional structure of claim 1, wherein the epithelial cells are airway epithelial cells.

6. The three-dimensional structure of claim 4, wherein the epithelial cells are selected from ciliated cells, secretory cells, ionocytes, basal cells, submucosal gland cells, club cells, Type I and Type II alveolar cells, hillock cells, or any combination thereof.

7. The three-dimensional structure of claim 4, wherein the epithelial cells comprise lung cells, bronchial cells, tracheal cells, alveolar cells, mammary cells, kidney cells, bladder cells, corneal cells, prostate cells, renal cells, vaginal cells, cervical cells, intestinal cells, or combinations thereof.

8. The three-dimensional structure of claim 1, wherein the three-dimensional structure comprises an interior chamber.

9. The three-dimensional structure of claim 8, wherein the interior chamber comprises a plurality of cells of a third cell type.

10. The three-dimensional structure of claim 9, wherein the third cell type of cells comprises stromal cells, mesenchymal cells, chondrocytes, osteoblasts, adipocytes, myocytes, pericytes, endothelial cells, or any combination thereof.

11. The three-dimensional structure of claim 10, wherein the stromal cells comprise fibroblasts, myofibroblasts, adipocytes, fibrocytes, pericytes, mesenchymal stem cells, macrophages, mast cells, lymphocytes, neutrophils, other leukocytes, endothelial cells, smooth muscle cells, or any combination thereof.

12. The three-dimensional structure of claim 1, wherein the diameter of the three-dimensional structure is between about 100 micrometers and about 5 mm.

13. The three-dimensional structure of claim 1, wherein, when the diameter is altered, the diameter is reduced by between about 10% and about 60% resulting in a second diameter, or the diameter is reduced by a decrease in roundness resulting in a second diameter, or a combination thereof.

14. The three-dimensional structure of claim 1, wherein the ratio amount of the second cell type and the first cell type is between 100:1 and 1:1.

15. The three-dimensional structure of claim 1, wherein the first cell type, the second cell type, and/or the third cell type comprise human cells.

16. A method of modeling Bronchiolitis Obliterans Syndrome, the method comprising:
providing the three-dimensional structure of claim 1, and
exposing the exterior layer of the three-dimensional structure to a plurality of blood cells, under conditions effective to model Bronchiolitis Obliterans Syndrome.

17. The method of claim 16, wherein the method is carried out after a lung transplant or after a bone marrow transplant.

18. The method of claim 16, wherein the first cell type comprises stromal cells.

19. The method of claim 18, wherein the stromal cells comprise fibroblasts.

20. The method of claim 16, wherein the second cell type comprises epithelial cells.

21. The method of claim 20, wherein the epithelial cells are airway epithelial cells.

22. The method of claim 20, wherein the epithelial cells are selected from ciliated cells, secretory cells, ionocytes, basal cells, submucosal gland cells, club cells, Type I and Type II alveolar cells, hillock cells, or any combination thereof.

23. The method of claim 22, wherein the epithelial cells comprise lung cells, bronchial cells, tracheal cells, alveolar cells, mammary cells, kidney cells, bladder cells, corneal cells, prostate cells, renal cells, vaginal cells, cervical cells, intestinal cells, or combinations thereof.

24. The method of claim 16, wherein the three-dimensional structure comprises an interior chamber.

25. The method of claim 16, wherein the interior chamber comprises a plurality of cells of a third cell type.

26. The method of claim 25, wherein the third cell type of cells comprises stromal cells, mesenchymal cells, chondrocytes, osteoblasts, adipocytes, myocytes, pericytes, endothelial cells, or any combination thereof.

27. The method of claim 26, wherein the stromal cells comprise fibroblasts, myofibroblasts, adipocytes, fibrocytes, pericytes, mesenchymal stem cells, macrophages, mast cells, lymphocytes, neutrophils, other leukocytes, endothelial cells, smooth muscle cells, or any combination thereof.

28. The method of claim 25, wherein the first cell type, the second cell type, and/or the third cell type comprise human cells.

29. The method of claim 16, wherein the plurality of blood cells comprises one or more of hematopoietic stem cells, hematopoietic circulating cells, or other leukocytes, peripheral blood stem cells (PBSCs), patient-derived cells, engineered cells, peripheral blood mononuclear cells (PBMCs), isolated lymphocytes, chimeric antigen receptor (CAR)-T cells, neutrophils, or monocytes.

30. The method of claim 16, wherein the method is carried out in vitro.

31. The method of claim 16 further comprising:
providing one or more healthy lung cells and one or more healthy donor HLA-mismatched immune cells.

32. The method of claim 16 further comprising:
identifying one or more of a plurality of disease progression phases.

33. The method of claim 32 further comprising:
testing a treatment during at least one of the one or more of the plurality of disease progression phases.

34. The method of claim 33, wherein said treatment is selected from one or more of a cytokine blockade, one or more of a specific cell population blockade, one or more of an agent to aid in epithelia repair, or any combination thereof.

35. A method for assessing presence of or risk of developing a physiological condition, the method comprising:
providing the three-dimensional structure of claim 1, and
exposing the exterior layer of the three-dimensional structure to a plurality of blood cells, under conditions effective to assess presence of or risk of developing a physiological condition.

36. The method of claim 35, wherein the plurality of blood cells comprises one or more of hematopoietic stem cells, hematopoietic circulating cells, or other leukocytes, peripheral blood stem cells (PBSCs), patient-derived cells, engineered cells, peripheral blood mononuclear cells (PBMCs), isolated lymphocytes, chimeric antigen receptor (CAR)-T cells, neutrophils, or monocytes.

37. The method of claim 35, wherein the plurality of blood cells are present in the amount of between about 300 to about 300,000 cells.

38. The method of claim 35, wherein the physiological condition comprises a recapitulation of one or more diseases involving an immune response.

39. The method of claim 35, wherein the physiological condition comprises one or more diseases involving epithelial-stromal-blood cells.

40. The method of claim 35 further comprising:
varying degrees of human leukocyte matching of the three-dimensional structure for at least one of the cells of the first cell type, the second cell type, and/or the blood cells.

41. The method of claim 40, wherein the varying degrees of human leukocyte matching for at least one of the cells of the three-dimensional structure comprises varying the degree of antigen matching.

42. The method of claim 40, wherein the varying degrees of human leukocyte matching for at least one of the cells of the three-dimensional structure comprises varying the degree of protein level.

43. The method of claim 40, wherein the varying degrees of human leukocyte matching comprises fully matching human leukocyte antigens (HLA) between one or more cells of the second cell type and one or more blood cells.

44. The method of claim 40, wherein the varying degrees of matching comprises tuning human leukocyte antigens (HLA) mismatch between one or more cells of the second cell type and one or more blood cells.

45. The method of claim 35 further comprising:
culturing a plurality of the three-dimensional structure in a plurality of wells in a single-organoid-per-well format; and
manipulating a growth factor level in the plurality of wells.

46. The method of 45 further comprising:
isolating RNA from the single three-dimensional structure.

47. The method of 46 further comprising:
sequencing RNA isolated from the single three-dimensional structure.

48. The method of 46 further comprising:
harvesting a supernatant from the plurality of wells; and
assaying the supernatant.

49. A method of identifying one or more biomarkers of a disease, the method comprising:
providing the three-dimensional structure of claim 1;
exposing the exterior layer of the three-dimensional structure to a plurality of blood cells; and
identifying one or more biomarkers of a disease.

50. The method of claim 49 further comprising:
diagnosing or prognosing a disease based on the presence or absence of one or more biomarkers of disease.

51. The method of claim 50, wherein the biomarkers of disease are selected from the group consisting of interleukin-6 (IL-6), c-reactive protein (CRP), tumor necrosis factor-alpha (TNF-α), donor-derived cell-free DNA (dd-cfDNA), lymphocyte count, neutrophil count, surfactant proteins (SP-A, SP-D), krebs von den Lungen-6 (KL-6), matrix metalloproteinases (MMP), transforming growth factor-beta (TGF-β), procalcitonin (PCT), galactomannan, cytomegalovirus (CMV) DNA, pathogen-specific PCR, b-type natriuretic peptide (BNP), lactate dehydrogenase (LDH), total protein, albumin, erythrocyte sedimentation rate (ESR), and fibrinogen.

52. The method of claim 49 further comprising:
identifying targetable pathways for treatment of the disease.

53. The method of claim 52, wherein the targetable pathway for treatment of the disease is mechanistic target of rapamycin (mTOR) pathway in T cells.

54. The method of claim 49 further comprising:
testing a response of the one or more biomarkers of a disease in response to one or more interventions.

55. The method of claim 54, wherein the one or more interventions is a TNF-alpha blockade.

56. A method of making a three-dimensional structure, the method comprising:
providing a plurality of cells of a first cell type;
providing a plurality cells of a second cell type;
providing a basement membrane matrix material; and
co-culturing the plurality of cells of the first type, the plurality of cells of the second cell type, and the basement membrane matrix material under conditions effective to form the three-dimensional structure of claim 1.
